(19) United States
(12) Patent Application Publication
(10) Pub. No.: US 2013/0040285 A1
(43) Pub. Date: Feb. 14, 2013

(54) Saliva Polymerase Chain Reaction Assay for Cytomegalovirus Infection

(71) Applicants: Suresh B. Boppana, Birmingham, AL (US); Shannon Ross, Vestavia, AL (US); Zdenek Novak, Hoover, AL (US)

(72) Inventors: Suresh B. Boppana, Birmingham, AL (US); Shannon Ross, Vestavia, AL (US); Zdenek Novak, Hoover, AL (US)

(73) Assignee: The UAB Research Foundation, Birmingham, AL (US)

(21) Appl. No.: 13/581,600

(22) PCT Filed: May 2, 2011
(86) PCT No.: PCT/US2011/034809

§ 371 Date: Sep. 20, 2012

Related U.S. Application Data
(60) Provisional application No. 61/329,897, filed on Apr. 30, 2010.

(51) Int. Cl. C12Q 1/70

(52) U.S. Cl. 435/5

(57) ABSTRACT

Congenital cytomegalovirus (CMV) infection is an important cause of disease in infants and immune-compromised adults. Most infants infected with CMV are not promptly identified because of the absence of symptoms. It has been discovered that an assay based on the polymerase chain reaction (PCR) for testing of saliva specimens for CMV is rapid, accurate, and inexpensive. Some versions of the assay employ primers that are specific for sequences flanking the highly conserved major envelope glycoprotein B or the highly conserved immediate early 2 exon 5 genes. The assay exceeds the standard rapid culture technique in accuracy, speed, and economy. When the assay is performed on dried saliva, it loses no significant amount of accuracy, and is surprisingly much more sensitive than a PCR assay using dried blood. This assay will permit broader testing to detect and intervene in congenital CMV infection, potentially avoiding numerous cases of associated disease.

### FIELD

The field of the disclosure is assays to detect nucleic acids, particularly those to detect nucleic, acids indicative of the presence of a pathogen in a biological sample.

### BACKGROUND

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Cytomegalovirus (CMV) is one of the herpesviruses. This group of viruses includes the herpes simplex viruses, varicella-zoster virus (which causes chickenpox and shingles), and Epstein-Barr virus (which causes infectious mononucleosis, also known as mono), CMV is a common virus that infects people of all ages. Most people with CMV infections have no signs or symptoms. However, CMV can cause disease in people with a weakened immune system such as transplant patients, people infected with HIV, and newborns: newborn babies are by far the largest group vulnerable to CMV, About 1 in 150 children is born with congenital CMV infection.

CMV can be transmitted from a pregnant woman to her fetus during pregnancy. The virus in the mother's blood crosses over the placenta and infects the fetus' blood. Most babies with congenital CMV infection never have health problems. But in some babies, congenital CMV infection causes health problems that may be apparent at birth or may develop later during infancy or childhood. In some cases, serious symptoms of CMV infection are present at birth, such as prematurity, liver problems, lung problems, spleen problems, low birth weight, small head size (microecphaly), and seizures, Congenital CMV can cause chronic illness, leading to hearing loss, vision loss, mental disability, small head size, lack of physical coordination, seizures, and death. CMV is a leading non-genetic cause of sensorineural hearing loss (SNHL). About 10% to 15% of children born with congenital CMV infection develop SNHL, although most do not exhibit symptoms at birth.

At the current time, it is not possible to identify most of the infants with asymptomatic congenital CMV infection at risk for CMV-associated SNHL and other diseases because of the lack effective methods to diagnose CMV in asymptomatic children at birth. If early identification of congenitally infected infants were available it would allow for targeted monitoring and intervention during critical stages of development.

Previous methods of diagnosing congenital CMV infection have relied on the ability to cultivate the virus in cultures of human cells. Virus isolation in saliva or urine using traditional cell culture assays and, more recently, centrifugation-enhanced rapid culture assays, have been considered standard methods for the diagnosis of congenital CMV infections. Saliva rapid culture assay has been demonstrated to be >98% sensitive in identifying infants with congenital CMV infection (Balcarek, 1993; Boppana et al., JAMA 303(14):1375-1382 (2010)). However, culture-based assays are not suitable for mass screening of newborns because they are not amenable to automation, are labor intensive, and require tissue culture facilities.

A more promising method for detecting CMV in newborns is the polymerase chain reaction (PCR), which can detect specific DNA sequences in very low amounts quickly and accurately. Strategies utilizing dried blood spot PCR assays for widespread newborn screening were initially considered because earlier studies demonstrated that infants with congenital CMV infection can be identified by dried blood spot PCR assays, and because dried blood spot specimens are collected routinely from all newborns. However, the findings of a multicenter newborn CMV screening study comparing dried blood spot PCR to the standard rapid culture of saliva specimens demonstrated that dried blood spot PCR assays lack sufficient sensitivity to detect most congenital CMV infections (Boppana, 2010).

### SUMMARY

There is a long-felt but unmet need in the art for a rapid, accurate, and inexpensive method to detect congenital CMV infection, particularly in asymptomatic newborns. This goal, as well as others, is served by various embodiments of the methods and kits provided in this disclosure (although it is to be understood that not all such goals will be fulfilled by every embodiment disclosed).

Methods are provided for detecting viruses in samples. A general embodiment of the method comprises contacting the sample with a first forward amplification primer and a first reverse amplification primer, said primers hybridizing under highly stringent conditions to sequences flanking a first target sequence, to form a reaction mixture; performing the polymerase chain reaction on the reaction mixture to form an amplicon; and detecting the target sequence in the amplicon; wherein the sample is not subject to DNA extraction prior to performing the polymerase chain reaction. An alternative general embodiment of the method comprises contacting the sample with a first forward amplification primer and a first reverse amplification primer, said primers hybridizing under highly stringent conditions to sequences flanking a target sequence; contacting the sample with a second forward amplification primer and a second reverse amplification primer, said second primers hybridizing under highly stringent conditions to sequences flanking a second target sequence, to form a reaction mixture; performing the polymerase chain reaction on the reaction mixture to form an amplicon; and detecting the target sequence in the amplicon. In certain embodiments, the target sequence in the amplicon is detected through the use of a probe that hybridizes specifically to the target sequence under highly stringent conditions. Additional forward and reverse primers may be added as desired to amplify additional targets within the amplicon.

A particular embodiment of the method comprises contacting a saliva sample with a first forward amplification primer and a first reverse amplification primer, said primers hybridizing under highly stringent conditions to the major envelope glycoprotein B gene or a portion thereof; contacting the sample with a second forward amplification primer and a second reverse amplification primer, said second primers hybridizing under highly stringent conditions to sequences flanking the immediate early region 2 (IE-2) exon 5 or a portion thereof; to form a reaction mixture; performing real-time polymerase chain reaction on the reaction mixture to form an amplicon; contacting the amplicon with a probe specific for the amplified region of glycoprotein B under highly stringent conditions; contacting the amplicon with a probe specific to the amplified regions of IE-2 exon 5 under highly stringent conditions; and detecting any hybridized probe.

Kits for the detection of a virus in saliva by real-time PCR are provided. In a general embodiment, the kit comprises a first forward primer, a first reverse primer, a DNA polymerase, and does not include reagents for DNA extraction. In an alternative general embodiment, the kit comprises a first forward primer, a first reverse primer, a second forward primer, a second reverse primer, and a DNA polymerase. In a particular embodiment, the kit is for the detection of human CMV in a saliva sample, the first primers are specific for the major envelope glycoprotein B or a portion thereof, the second primers are specific for the immediate IE-2 exon 5 or a portion thereof; the kit further comprises a first probe specific for the amplified region of the major envelope glycoprotein B, and the kit further comprises a second probe specific for the IE-2 exon 5.

### BRIEF DESCRIPTION OF THE DRAWINGS

FIG. 1A diagram illustrating prospective screening study of 34,989 newborns to determine the sensitivity and specificity of saliva real-time PCR assays in identifying infants with congenital CMV infection.

FIG. 2: A simplified map of the human cytomegalovirus genome. UL—unique long, US—unique short, TRL—terminal repeat long, IRL—internal repeat long (inverted repeat of TRL), TRS—terminal repeat short, IRS—internal repeat short (inverted repeat of TRS), From NIST, citing Kotenko et al. PNAS 97:1695-1700 (2000).

FIG. 3: A detailed map of the human cytomegalovirus genorne. The green arrows represent previously annotated ORFs of HCMV deemed likely to encode a bona fide polypeptide, red arrows designate ORFs deemed unlikely to encode a polypeptide, and blue arrows indicated previously unrecognized ORFs that the author's analysis predicts have high potential to encode proteins. The gray box marks the additional sequence found in the HCMV Toledo strain, locating it with respect to the AD169 genome. Rectangles superimposed on the line represent the sequence-identify terminal repeats. Each mark on the sequence line represents 1,000 bp. From NIST, citing Murphy et al. PNAS 100 (2003).

FIG. 4: Organization of open reading frames in several strains of human CMV. (A) Conventional ORF maps of the AD169 laboratory strain and clinical isolates. From the left, the AD169 genome contains TRL1-14 (green arrow), UL1-132 (dark blue arrow), IRL14-1 (green arrow), IRS1 (red arrow), US1-36 (light blue arrow), and IRS1 (red arrow). From the left, clinical isolates contain a unique domain including R1:1-14 and UL1-151 (green plus dark blue arrow) followed by IRS1 (red arrow), US1-36 (light blue arrow), and TRSI (red arrow). (B) ORF maps of the BAC clones whose sequences are reported in Murphy et al. (see citation below). Sequences were linearlized at the position corresponding to nucleotide 1 of the original AD169 sequence. Arrows indicate the relative orientations of the repeated and unique ORF blocks. The RL region is not repeated in the clinical isolates (Toledo, PH, TR, and FIX); there is a single copy of the region (green segment appended to the unique long region). The Towne laboratory strain contains a block of ORFs (UL147-154, orange arrow) that is not present in AD169, and the clinical isolates contain a block of ORFs (UL133-151, orange arrow) that is not present in the AD169 laboratory strains. The BAC inserts are identified (B), and viral ORFs deleted during BAC insertions are listed in parentheses. From NIST, citing Murphy et al. PNAS 100 no 25 (2003).

### DETAILED DESCRIPTION

### Definitions

The terms “comprise”, “comprising”, “comprised”, “comprises”, and variants thereof as used herein means that the structures, steps, components, or other elements following the term are included in an embodiment without excluding any other such structures, steps, components, or other elements that may be present. Such terms shall not be construed to limit the embodiments except regarding the inclusion of the structures, steps, components, or other elements following the term.

The term “highly stringent conditions” as used herein means that the conditions of temperature and ionic strength are selected so that it enables hybridization to be maintained between two complementary nucleic acid fragments. Such conditions can also be affected by the presence of certain enzymes, notably helicase. These conditions are well known by the person skilled in the art, and are described, for example, in the book by Sambrook at el. Molecular Cloning, a Laboratory Manual. Third Edition. CSHL press, Cold Spring Harbor, N.Y., 2001, which is incorporated herein by reference to teach how to achieve such conditions. A person of ordinary skill in the art would be able to fluctuate the various factors involved to achieve the desired level of stringency for a given pair of complementary DNA fragments with a known melting point.

Typically, hybridization of two strands at high stringency requires that the sequences exhibit a high degree of complementarity over an extended portion of their length. Examples of high stringency conditions include: hybridization to filter-bound DNA in 0.5 M NaHPO4, 7% SDS, 1 mM EDTA at 65° C., followed by washing in 0.1×SSC/0.1% SDS (where 1×SSC is 0.1.5 M NaCl, 0.15 M Na citrate) at 68° C. or for oligonucleotide molecules washing in 6×SSC/0.5% sodium pyrophosphate at about 37° C. (for 14 nucleotide-long oligonucleotides), at about 48° C. (for about 17 nucleotide-long oligonucleotides), at about 55° C. (for 20 nucleotide-long oligonucleotides), and at about 60° C. (for 23 nucleotide-long oligonucleotides). In a specific embodiment, the highly stringent conditions are those described in the example below.

Conditions of intermediate or moderate stringency (such as, for example, an aqueous solution of 2×SSC at 65° C.; alternatively, for example, hybridization to filter-bound DNA in 0.5 NI NaHPO4, 7% SDS, 1 mM EDTA at 65° C. followed by washing in 0.2×SSC/0.1% SDS at 42° C.) and low stringency (such as, for example, an aqueous solution of 2×SSC at 55° C.), require correspondingly less overall complementarity for hybridization to occur between two sequences. Specific temperature and salt conditions for any given stringency hybridization reaction depend on the concentration of the target DNA and length and base composition of the probe, and are normally determined empirically in preliminary experiments, which are routine (see Southern, J. Mol. Biol. 1975; 98:503; Sambrook et al., Molecular Cloning: A Laboratory Manual, 2nd ed., vol, 2, ch. 9.50, CSH Laboratory Press, 1989; Ausubel et al. (eds.), 1989, Current Protocols in Molecular Biology, Vol. 1, Green Publishing Associates, Inc., and John Wiley & Sons, Inc., New York, at p. 2.10.3).

As used herein, the term “standard hybridization conditions” refers to hybridization conditions that allow hybridization of two nucleotide molecules having at least 50% sequence identity. According to a specific embodiment, hybridization conditions of higher stringency may be used to allow hybridization of only sequences having at least 75% sequence identity, at least 80% sequence identity, at least 90% sequence identity, at least 95% sequence identity, or at least 99% sequence identity.

The term “individual”, “subject” or “patient” as used herein refers to any animal, including birds or mammals, such as mice, Norway rats, cotton rats, gerbils, cavies, hamsters, other rodents, rabbits, dogs, cats, swine, cattle, sheep, goat, horses, domestic fowl, primates, and humans. The term may specify male or female or both, or exclude male or female.

The term “including” as used herein is non-limiting in scope, such that additional elements are contemplated as being possible in addition to those listed; this term may be read in any instance as “including, but not limited to.”

The term “wild-type” as used herein refers to an allele of a gene that is capable of providing the normal functioning of the gene and is the predominant allele in the population.

The term “mutant” as used herein applied to an allele refers to an allele that is not wild-type. Some mutant alleles are phenotypically similar or indistinguishable from the wild-type, while other mutant alleles produce a phenotype that is easily distinguished from that associated with the wild-type.

In all cases the use of feminine pronouns should be construed to include the masculine, the use of masculine pronouns should be construed to include the feminine, the use of the singular should be construed to include the plural, and the use of the plural should be construed to include the singular, unless explicitly stated otherwise. When an element or a step is described in this disclosure, it is to be understood that the element may be embodied in multiple structures or the step may be embodied in multiple acts unless stated otherwise; in a similar vein, when multiple elements or steps are described in this disclosure, it is to be understood that the elements may be embodied in a single structure or the steps may be embodied in a single act unless stated otherwise.

Methods of Detecting Viruses

A method for the detection of viruses is provided herein. The method comprises performing a genetic analysis on a sample. Some embodiments of the method comprise detecting CMV in a sample using PCR. Some such embodiments of the genetic analysis comprise the polymerase chain reaction utilizing primers that will hybridize under highly stringent conditions with at least one of a nucleic acid consensus sequence for CMV, a sequence unique to CMV, or a region flanking such a sequence.

The sample may be any material that is suspected to contain the virus. Examples of such samples include a biological sample, a sample from a subject, an environmental sample, and a culture sample. In some embodiments the sample is a biological sample from a subject, including a fluid or tissue sample. The fluid or tissue may be any that is known or suspected to harbor the virus. The subject may be any organism that is known or suspected to harbor the virus, including Homo sapiens. In some embodiments of the method, the subject is a human neonate. Some embodiments of the sample comprise a saliva sample, for example obtained from a cheek swab. In further embodiments the sample is dried to stabilize the sample during storage or transportation. In other embodiments of the method, the sample is stored in a liquid medium, including a sucrose/phosphate transport medium (TM). The method may be an in vitro method, although in vivo embodiments are conceivable.

The virus may be any virus suspected to exist in the sample. In some embodiments of the method the virus is a virus that is found in saliva. In further embodiments the virus is CMV. The CMV may be a human CMV or a CMV found in another animal. In particular embodiments the CMV is a specific strain of human CMV.

Once the sample suspected of harboring the virus has been obtained, it can be analyzed. The analysis may be performed using any techniques known in the art including, but not limited to, sequencing, PCR, RT-PCR, quantitative PCR, restriction fragment length polymorphism, hybridization techniques, Northern blot, microarray technology, and similar techniques. In determining the expression level of a gene or genes in a genetic sample, the level of expression may be normalized by comparison to the expression of another gene such as a well known, well characterized gene or a housekeeping gene. In detecting the presence of an expressed target sequence, reverse-transcriptase PCR(RT-PCR) can be used to detect the presence of a specific nRNA population in a complex mixture of thousands of other mRNA species. Hybridization to clones or oligonucleotides arrayed on a solid support (i.e., gridding) can be used to both detect the presence of and quantitate the level of a target sequence. In this approach, a target sequence complementary to a sequence encoding a target sequence (which may be referred to as an analyte) to be detected is fixed to a substrate. The substrate may be of any suitable type including but not limited to glass, nitrocellulose, nylon or plastic. At least a portion of the complementary nucleic acid is attached to the substrate and then incubated with the analyte, isolated from the sample of interest. Hybridization between the substrate bound nucleic acid and the analyte can be detected and quantitated by several means including but not limited to radioactive labeling or fluorescence labeling of the analyte or a secondary molecule designed to detect the hybrid. Quantitation of the target sequence can be done by comparison of the intensity of the signal from the analyte compared with that determined from known standards.

The polymerase chain reaction is well known to those of ordinary skill in the art, and is taught in its basic form by Sambrook et al., Molecular Cloning: A Laboratory Manual. 3^{rd} Ed., Cold Spring Harbor Press, 2001, which is incorporated herein by reference to teach this technique. Variants of PCR may be used, including real-time (quantitative) PCR. Other suitable variant of PCR may be used with the assay, including quantitative PCR, multiplex PCR, nested PCR, multiplex ligation-dependent probe amplification, miniprimer PCR, and hot-start PCR. Loop-mediated isothermal amplification (LAMP) can be used, as described by Notoni et al., Nucleic Acid Res. 28:e63 (2000); LAMP has been used successfully to detect CMV in Vitreous fluid, for example (Reddy et al., J. Clinical Microbiol., 48(6):2050-2052 (2010)). Real-time PCR has the advantage of allowing direct and immediate observation of target amplification, enhancing the sensitivity of the method and reducing the time required.

In selecting the primers for PCR, the specificity of the hybridization between the primer and the target sequence can be adjusted by increasing or decreasing the length of the primer. In general, a primer will give increased specificity and differentiation as a mismatch between the target sequence and the primer will have a significant impact on the hybridization efficiency. A longer primer will provide less specificity but greater hybridization efficiency and therefore increased sensitivity. The nature of the primer will influence the composition of the primer. One of ordinary skill in the art would be able to alter the parameters of the primer to achieve the desired specificity and sensitivity of binding of the primer to the target sequence. In one embodiment, the primer is 10 to 50 base pairs in length, in an alternate embodiment, the primer is 2040 base pairs in length. In yet another alternate embodiment the primer is 15 to 26 base pairs in length.

The primers may also be varied by incorporating one or more locked nucleic acids (LNA). LNA are a class of nucleic acids containing altered nucleosides whose major distinguishing characteristic is the presence of a methylene bridge between the 2′-0 and 4′C atoms of the ribose ring. LNA nucleosides containing the five common nucleobases that appear in DNA and RNA (A, T, U, C, O) can base-pair with their complementary nucleosides according to Watson-Crick rules. The molecular differences between normal nucleosides and LNAs give rise to differences in the stability of nucleic acid duplexes formed between LNA containing nucleic acids and non-LNA containing nucleic, acids. Typically, each LNA nucleotide incorporated increases the Tₘ of a LNA/DNA nucleotide complex by 2-6° C. as compared to a corresponding DNA/DNA complex.

The primers used in the PCR will hybridize with at least one of a nucleic acid consensus sequence for all strains of the virus (for example, CMV), a nucleic, acid consensus sequence for a given strain of the virus, a sequence unique to the virus, or a sequence flanking any of the foregoing. In this context a “consensus sequence” is a sequence that is substantially conserved within a taxonomic group of viruses, such as CMV or human CMV. The sequence need not occur at the same location in the genome in each case. It is understood in the art that occasional mutations occur in consensus sequences, without negating the status of the mutant sequence as a consensus sequence. One example of such a mutation is a “silent mutation,” in which a nucleotide base substitution in an open reading frame does not change the amino acid specified by the corresponding mRNA codon. An exemplary silent mutation is a DNA substitution from TTT to TTC. As both DNA codons encode phenylalanine, this mutation has no effect on the final protein. Consensus sequences may vary at some loci by definition, in which case notations such as [CT] (either C or T), N (any base), Y (any pyrimidine), and R (any purine) are used to indicate variation. A consensus sequence may in some cases be defined to allow a certain minimum amount of homology to a specified sequence, for example 99.9%, 99.5%, 98%, 97%, 96%, 95%, 90%, 85%, 80%, 75%, and 70%.

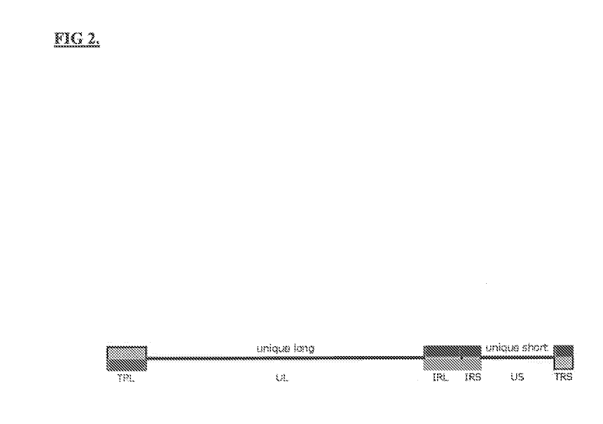

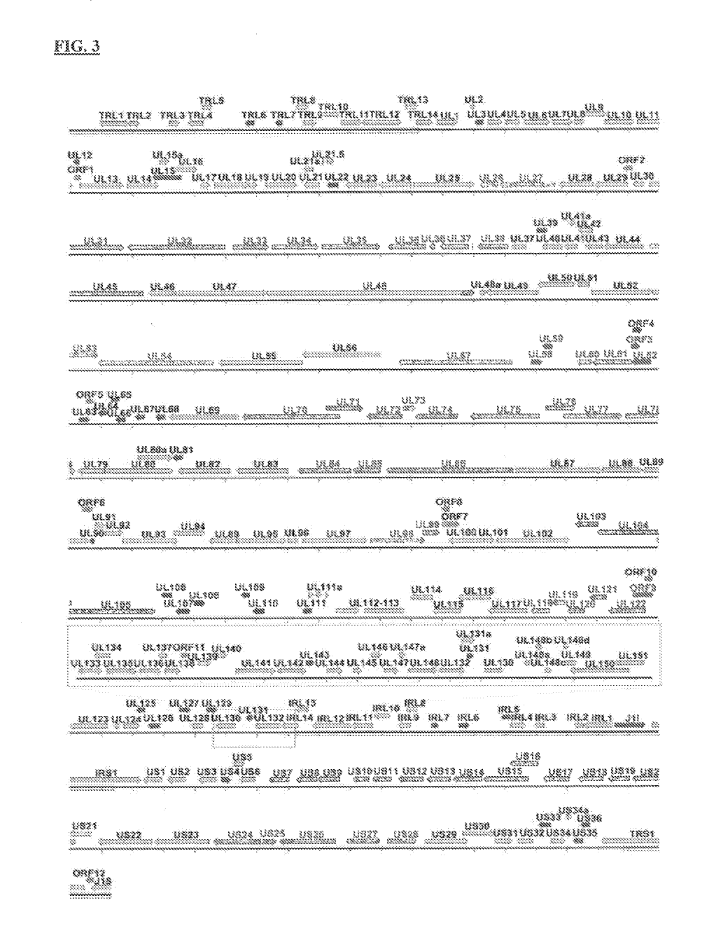

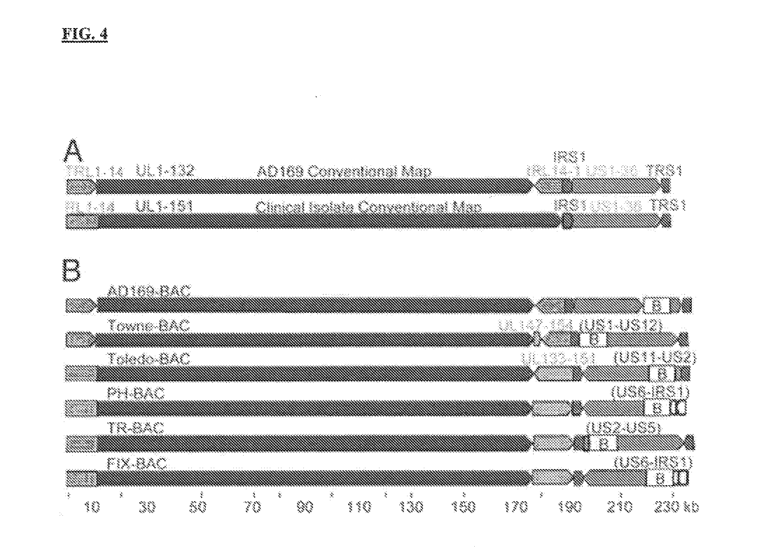

Human CMV is extremely well characterized genetically. An overall genomic map has been developed and is publicly provided online by the United States National Institute of Standards and Technology (http://www.nist.govimml/biochernicallgerietics/cmv_structure.cfm). The simplified map of the CMV genome is reproduced in FIG. 2, and the detailed map of the CMV genome is reproduced in FIG. 3. FIG. 4 illustrates the organization of open reading frames in human CMV. The CMV genome is organized as two regions of unique sequences, unique long (UL) and unique short (US), flanked by two sets of inverted repeats (TRUIRL) and (IRS/TRS) (light shaded boxes). The full genomes of at least 15 strains have been completely sequenced and are publicly available. The consensus sequence can be determined by analyzing the literature of known genomic sequences of the virus or variants thereof. The strains for which full sequences exist include strain Towne (GenBank accession AY31.51.97.2), strain 3157 (GenBank accession GQ221974.1), strain 3301 (GenBank accession GQ466044.1), strain AD169 substrain varUC (GenBank accession FJ527563.1), strain AD169 substrain varUK (GenBank accession BK000394.5), strain AF1 (GenBank accession GU179291.1), strain HAN13 (GenBank accession GQ221973.1), strain HAN20 (GenBank accession GQ396663.1), strain 1-JAN38 (GenBank accession GQ396662.1), strain JP (GenBank accession GQ221975.1), strain Toledo (GenBank accession GU937742.1), strain U8 (GenBank accession GU179288.1), strain U11 (GenBank accession GU179290.1), strain VR1814 (GenBank accession GUI 79289.1), and strain Mer1 in (transgenic) (GUI 79001.1). All of the foregoing genomic sequences are incorporated herein by reference. The genome of human CMV has been analyzed repeatedly using several techniques (Rigoutsos et al., J. Virol. 77:4326-4344 (2002); Dunn et al., PNAS 100:14223-14228 (2003); Cunningham et al., J. Gen. Viral. 91:605-615 (2010); Davison et al., J. Gen. Virol. 84:17-28 (2003); Cha et al., J. Virol, 70:78-83 (1996)). Using sequence analysis tools widely known in the art a person of ordinary skill will be able to identify consensus sequences without undue experimentation. For example, the publicly available BLAST sequence alignment tool will quickly locate areas of identity between all genetically characterized strains (or any subset thereof).

Sequences unique to a given virus can be identified by analyzing the literature of known genomic sequences of the virus or variants thereof. Public databases of genetic information can be queried for sequences from other sources containing regions of identity to genomic sequences of the virus. Any genomic sequences of the virus that have no identical matches can be used as unique sequences in the method.

Some embodiments of the unique sequence are also consensus sequences for the virus. Such sequences have the advantage of always being present in the analyte virus, but never being present in other sources of nucleic acids. This will eliminate or reduce both false negative results (when a strain of the analyte virus is present in the sample that lacks the target sequence) and false positive results (when the target sequence is present, but the analyte virus is not).

Examples of the target sequences for CMV include the glycoprotein B gene, the AD-1 region of glycoprotein B gene (or a nearby region), and the immediate IE-2 exon 5 gene. In further embodiments the forward primer the major envelope glycoprotein B is AGO TCT TCA AGG AAC TCA (ICA AGA (SEQ ID NO: 1), and the reverse primer is COG CAA TCG GTT TGT TGT AAA (SEQ ID NO: 2). In yet further embodiments, forward primer for the IE-2 exon 5 gene is GAG CCC GAC TTT ACC ATC CA (SEQ ID NO: 4), and the reverse primer is CAG CCG GCG GTA TCG A (SEQ ID NO: 5).

Glycoprotein B is a highly conserved component of the complex cell-entry machinery of herpes viruses. In CMV, the glycoprotein B gene is generally located at positions 82,066-84,789 of the genome. The general consensus sequence of the glycoprotein B gene in human CMV is shown in the attached sequence listing as SEQ ID NO: 8. The antigenic determinant (AD) 1 region of the gene is generally located at positions 1-206 of SEQ ID NO: 8. The target may be the glycoprotein B gene, the AD-1 region of glycoprotein B, a region nearby the AD-1 region, or a variant of either comprising conservative substitutions. In a particular embodiment the target is SEQ ID NO: 8, positions 1-206 of SEQ ID NO: 8, positions 275-346 of SEQ ID NO: 8, the glycoprotein B gene from human CMV strain Merlin (SEQ ID NO: 10), positions 1-206 of SEQ ID NO: 10, positions 275-346 of SEQ ID NO: 10, or a variant of any of the foregoing comprising a conservative substitution.

The immediate early (IE) gene region of human CMV encodes the first group of proteins to be expressed after the infection of a cell. It comprises two splicing regions, IE-1 and IE-2. Exon 5 of IE-2 is highly conserved, and is generally found at positions 170,689-174,090 in the human CMV genome. The general consensus sequence of IE-2 exon 5 is provided in SEQ ID NO: 7 of the attached sequence listing. The target may be IE-2 exon 5, or a variant thereof comprising conservative substitutions. In a particular embodiment the target is SEQ ID NO: 7, the IE-2 exon 5 gene from human CMV strain Merlin (SEQ ID NO: 10), or a variant of any of the foregoing comprising a conservative substitution.

A “conservative substitution” or “conservative amino acid substitution” is a substitution that would not be expected to change the functioning of the protein product of the gene. For example, a substitution in a DNA codon that codes for the same amino acid as did the original codon (due to the degeneracy of the DNA code) is one embodiment of a conservative substitution.

Such a substitution may involve a substitution of a native amino acid residue with a normative residue such that there is little or no effect on the polarity or charge of the amino acid residue at that position. Furthermore, any native residue in the polypeptide may also be substituted with alanine.

Such substitutions also encompass non-naturally occurring amino acid residues which are typically incorporated by chemical peptide synthesis rather than by synthesis in biological systems. These include peptidomimetics, and other reversed or inverted forms of amino acid moieties. It will be appreciated by those of skill in the art that nucleic acid and polypeptide molecules described herein may be chemically synthesized as well as produced by recombinant means.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

For example, conservative substitutions may involve the exchange of a member of one of these classes for a member from the same class. Such substituted residues may be introduced into regions of the derivatives that are homologous with corresponding genes in non-human CMV orthologs, or into the non-homologous regions of the molecule.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (Kyte et al., J. Mol. Biol., 157:105-131, 1982). It is known that certain amino acids may be substituted for other amino acids having a similar hydropathic index or score and still retain a similar biological activity. In making changes based upon the hydropathic index, the substitution of amino acids whose hydropathic indices are within +/−2 may be used; in an alternate embodiment, the hydropathic indices are with +/−1; in yet another alternate embodiment, the hydropathic indices are within +/−0.5.

It is also understood in the art that the substitution of like amino acids can be made effectively on the basis of hydrophilicity. The greatest local average hydrophilicity of a polypeptide as governed by the hydrophilicity of its adjacent amino acids, correlates with a biological property of the protein. The following hydrophilicity values have been assigned to amino acid residues: arginine (+3.0); lysine (+3.0); aspartate (+3.0.+-0.1); glutamate (+3.0,+-0.1); serine (+0.3); asparagine (+0.2); glutamine (+0.2); glycine (0); threonine (−0.4); proline (−0.5.+−,1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within +/−2 may be used; in an alternate embodiment, the hydrophilicity values are with +/−1; in yet another alternate embodiment, the hydrophilicity values are within +/−0.5.

Desired amino acid substitutions (whether conservative or non-conservative) can be determined by those skilled in the art at the time such substitutions are desired. For example, amino acid substitutions can be used to identify important residues of the target gene, or to increase or decrease the affinity of the target gene product with a particular binding target in order to increase or decrease the gene product's activity.

TABLE I

| Original Amino | | Preferred |
|---|---|---|
| Acid | Exemplary substitution | substitution |
| Ala | Val, Leu, Ile | Val |
| Arg | Lys, Gln, Asn | Lys |
| Asn | Glu | Glu |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Ile, Val, Met, Ala, Phe, Norleucine | Ile |
| Lys | Arg, 1,4-diaminobutyric acid, Gln, Asn | Arg |
| Met | Leu, Phe, Ile | Leu |
| Phe | Leu, Val, Ile, Ala, Tyr | Leu |
| Pro | Ala, Gly | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

Exemplary amino acid substitutions are set forth in Table 1. Some embodiments of the conservative substitution will comprise any of the above exemplary substitutions. Further embodiments will comprise one or more of the preferred substitutions listed in Table 1.

A skilled artisan will be able to determine suitable conservative variants using well known techniques. For identifying suitable areas of the molecule that may be changed without destroying activity, one skilled in the art may target areas not believed to be important for activity. With such a comparison, one can identify residues and portions of the molecules that are conserved among similar polypeptides. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a polypeptide that correspond to amino acid residues that are important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of that information, one skilled in the art may predict the alignment of amino acid residues of a polypeptide with respect to its three dimensional structure. One skilled in the art may choose not to make radical changes to amino acid residues predicted to be on the surface of the protein, since such residues may be involved in important interactions with other molecules. Moreover, one skilled in the art may generate test polypeptide derivatives containing a single amino acid substitution at each desired amino acid residue. The derivatives can then be screened using activity assays know to those skilled in the art and as disclosed herein. Such derivatives could be used to gather information about suitable substitution. For example, if one discovered that a change to a particular amino acid residue resulted in destroyed, undesirably reduced, or unsuitable activity, derivatives with such a change would be avoided. In other words, based on information gathered from such routine experiments, one skilled in the art can readily determine the amino acids where further substitutions should be avoided either alone or in combination with other mutations.

Numerous scientific publications have been devoted to the prediction of secondary structure from analyses of amino acid sequences (see Chou et al., Biochemistry, 13(2):222-245, 1974; Chou et al., Biochemistry, 113(2):211-222, 1974; Chou et al., Adv. Enzymol. Relat. Areas Mol, Biol., 47:45-148, 1978; Chou et al., Ann. Rev. Biochem., 47:251-276, 1979; and Chou et al., Biophys. J., 26:367-384, 1979). Moreover, computer programs are currently available to assist with predicting secondary structure of polypeptides. Examples include those programs based upon the Jameson-Wolf analysis (Jameson et al., Comput. Appl. Biosci., 4(1):181-186, 1998; and Wolf et al., Comput, Appl. Biosci., 4(1):187-191; 1988), the program PepPlot®, (Brutlag et al., CABS, 6:237-245, 1990; and Weinberger et al., Science, 228:740-742, 1985), and other new programs for protein tertiary structure prediction (Fetrow. et al., Biotechnology, 11:479-483, 1993).

Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptides or protein's structure (see Holm et al., Nucl. Acid. Res., 27(1):244-247, 1999).

Additional methods of predicting secondary structure include “threading” (Jones, D., Curr. Opin, Struct. Biol., 7(3):377-87, 1997; Suppl et al., Structure, 4(1):15-9, 1996), “profile analysis” (Bowie et al., Science, 253:164-170, 1991; Gribskov et al., Meth. Enzym., 183:146-159, 1990; and Gribskov et al., Proc. Nat. Acad. Sci., 84(13): 4355.

Other primers for the PCR-based detection of CMV include those disclosed in U.S. Patent Application Publication 2010/0297612, published on Nov. 25, 2010, by Slater et al. (see particularly the CMV primer/probe sets disclosed in Table 2 one pages 16-19). Recently Habbal et al., J. Med. Microbio. 58: 878-883 (2009), reviewed the literature describing PCR primers for the detection of human CMV and further evaluated 17 primer pairs experimentally, determining which known CMV strains are detected by which primer pairs and at what level of sensitivity. Numerous other primer sets (and appropriate probes) have been published, for example by Boeckh et al., J Clin Microbiol. 2004, 42(3): 1142-1148; Griscelli et al., J Clin Microbiol. 2001, 39(12): 4362-4369; Kalpoe et al., J Clin Microbiol. 2004, 42(4): 1498-1504; Leruez-VIIIe et al., J Clin Microbiol. 2003, 41(5):2040-2046; Machida et al., J Clin Microbiol, 2000, 38(7): 2536-2542; Tanaka et al., Journal of Medical Virology 2000, 60:455-462; Yun et al., Journal of Virological Methods 2003, 110(1): 73-79; Lengerova et al., Journal of Clinical Microbiology 2007, 45(3):1042-1044; Walter et al, Arch, Dis. Child, Fetal Neonatal Ed, 2008, 931:280-F285; Guiver et al., Transplantation 2001, 71(11): 1609-1615; Kearns et al., J Virol Methods 2001, 95:121-31; Ogawa et al., J Infect. Dis. 2007, 195(6):782-788. These disclosures are incorporated herein by reference only to teach such primers and/or probes and to enable one of ordinary skill in the art to use those primers and/or probes to detect CMV (or particular strains of CMV) without undue experimentation.

More than one primer set may be utilized to increase the specificity of the method; such embodiments would be expected to produce more than one amplification product in the amplicon. The second (or subsequent) forward and reverse primer may be any that are described as suitable as the first forward and reverse primers. It has been surprisingly discovered that amplifying two targets simultaneously significantly enhances the sensitivity of the method, in some embodiments of the method the first primer set flanks of the major envelope glycoprotein B, the AD-1 region of glycoprotein B, a region proximate to AD-1 region of glycoprotein B, or a portion thereof; and the second primer set flanks the IE-2 exon 5 gene or a portion thereof. In further embodiments the first forward primer comprises SEQ ID NO: 1, the first reverse primer comprises SEQ ID NO: 2, the second forward primer comprises SEQ ID NO: 4, and the second reverse primer comprises SEQ ID NO: 5.

Some embodiments of the assay further comprise contacting the PCR product (amplicon) with a probe that hybridizes with at least a portion of the amplified sequence. The probe comprises a nucleic acid sequence that hybridizes under highly stringent conditions with at least a portion of the amplified sequence(s) and a detectable tag. Detectable tags of various kinds are known in the art (colorimetric, fluorescent, isotopic, enzymatic, etc). Some embodiments of the probe specific for the major envelope glycoprotein B comprise the nucleic acid sequence AAC CCC TCA GCC ATT CTC TCG GC (SEQ ID NO: 3). Some embodiments of the probe specific for the IE-2 exon 5 gene comprise the nucleic acid sequence ACC GCA ACA AGA TT (SEQ ID NO: 6).

In selecting the nucleotide portion of the probe, the specificity of the hybridization between the probe and the target sequence can be adjusted by increasing or decreasing the length of the probe. In general, a probe will give increased specificity and differentiation as a mismatch between the target sequence and the probe will have a significant impact on the hybridization efficiency. A longer probe will provide less specificity but greater hybridization efficiency and therefore increased sensitivity. The nature of the primer will influence the composition of the primer. One of ordinary skill in the art would be able to alter the parameters of the probe to achieve the desired specificity and sensitivity of binding of the primer to the target sequence. In one embodiment, the probe is 10 to 50 base pairs in length. In an alternate embodiment, the probe is 20-40 base pairs in length. In yet another alternate embodiment the probe is 14 to 23 base pairs in length. The probe may also be varied by incorporating one or more locked nucleic acids (INA).

Some embodiments of the method do not require the extraction of DNA from the sample, and this step is omitted from such embodiments of the method. DNA extraction generally involves lysing any cells present in the same to release chromosomal DNA, removing lipids from the sample, removing protein from the sample, and precipitating nucleic acids from solution. It has been unexpectedly discovered that the detection of viruses in saliva by the methods provided does not require the extraction of DNA from the sample prior to amplification. This has the advantage of not requiring the time-consuming and laborious steps of DNA extraction.

Kits for Virus Detection

Kits for use with the assays are provided, comprising a polymerase, a forward primer, and a reverse primer. Embodiments of the kit may further comprise a probe. Further embodiments comprise at least one of a reagent for visualizing the probe (such as a substrate of horseradish peroxidase, or a radio-scintillation cocktail), or a source of illumination (such as an ultraviolet lamp) for visualizing the probe. The primers and probe may be any that are disclosed as suitable for the method. The polymerase may be any DNA polymerase, but it is preferably a DNA polymerase that is active at thermophilic temperatures to facilitate the rapid denaturation and hybridization of nucleic acids that occurs during PCR. Such thermophilic polymerases are well known in the art, and examples include DNA polymerase from the bacterium Thermus aquaticus. The kit may be for the detection of a virus in a saliva sample; in an exemplary embodiment the kit is for the detection of human CMV in a saliva sample. In further embodiments, the kit is for the diagnosis of CMV infection.

In a general embodiment, the kit is for the detection of a virus in saliva by real-time PCR, and includes no reagents for DNA extraction. In an alternative general embodiment, the kit comprises a first forward primer, a first reverse primer, a second forward primer, and a second reverse primer.

Some embodiments of the probe are specific to at least one of the major envelope glycoprotein B gene and the immediate early region 2 exon 5, Specific embodiments of the probe comprise at least one of SEQ ID NO: 3 and SEQ ID NO: 6. Of course, based on the public genomic data referred to above, one of ordinary skill in the art could design specific probes without undue experimentation.

In some embodiments of the kit, the primers hybridize under highly stringent conditions to sequences flanking the major envelope glycoprotein B gene, the AD-1 region, a region proximate to the AD-1 region, or a portion thereof. In more specific embodiments of the kit, the forward primer comprises SEQ ID NO: 1 and the reverse primer comprises SEQ ID NO: 2. In a particular such embodiment, the probe comprise SEQ ID NO: 3. In further embodiments of the kit, the primers hybridize under highly stringent conditions to sequences flanking 1E2 exon 5 or a portion thereof, In more specific embodiments of the kit, the forward primer comprises SEQ ID NO: 4 and the reverse primer comprises SEQ ID NO: 5. In a particular such embodiment, the kit of claim 40 further comprises a probe comprising SEQ ID NO: 6.

One exemplary embodiment of the kit comprises a first set of primers that hybridize under highly stringent conditions to the major envelope glycoprotein B gene or a portion thereof; a second set of primers that hybridize under highly stringent conditions to sequences flanking the 1E2 exon 5 or a portion thereof; a polymerase; and lacks reagents for DNA extraction from the sample.

The foregoing description illustrates and describes the methods, kits, and other teachings of the present disclosure. Additionally, the disclosure shows and describes only certain embodiments of the methods, kits, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the methods, kits, and other teachings of the present disclosure and to enable others skilled in the art to utilize the teachings of the present disclosure in such, or other, embodiments and with the various modifications required by the particular applications or uses. Accordingly, the methods, kits, and other teachings of the present disclosure are not intended to limit the exact embodiments and examples disclosed herein, All references cited herein are incorporated by reference as if fully set forth in this disclosure.

### EXAMPLE

### Proven Efficacy of Saliva PCR Assay

Summary of Methods:

In a prospective, multicenter newborn screening study, the results of a real-time PCR of liquid and dried saliva specimens were compared with rapid culture of saliva at birth.

Summary of Results:

Newborn CMV screening by rapid culture and PCR of saliva specimens identified a total of 177 of 34,989 (0.51%; 95% CI, 0.43%-0.59%) infants who were positive for CMV by one or more methods. Of 17,662 newborns screened using the liquid saliva PCR, all 85 (0.48%; 95% CI, 0.3W-0.59) culture-positive infants were also PCR-positive. Sensitivity and specificity of the liquid saliva PCR were 100% (85/85, 95% CI, 95.8%-100%) and 99.9 (17569/17577, 95% CI, 99.9%-100%), respectively. The positive and negative predictive values were 91.4% (85/93, 95% CI, 83.8%-96.2%) and 100% (17569/17569, 95% CI, 99.9%-100%), respectively. Of 17,327 newborns screened using the dried saliva PCR, rapid culture identified 76 (0.44%; 95% CI, 0.35-0.55%) as CMV infected and 74 of these infants were also identified by dried saliva PCR. Sensitivity and specificity of the dried saliva PCR assay were 97.4% (74/76, 95% CI, 90.8%-99.7%) and 99.9% (17245/17253, 95% CI, 99.9%-100%), respectively. The positive and negative predictive values were 90.2% (74/82, 95% CI, 81.7%-95.7%) and 99.9% (17243/17245, 95% CI, 99.9%-100%), respectively.

Summary of Conclusions:

Real-time PCR assays of both liquid and dried saliva specimens demonstrated high sensitivity and specificity for detecting CMV infection, and should be considered as potential newborn CMV screening tools.

Methods

Study Design: Infants born at seven hospitals in the U.S. between June 2008 and November 2009 were enrolled prospectively in the National Institute on Deafness and Other Communication Disorders (NIDCD) CMV and Hearing Multicenter Screening (CHIMES) Study. All live-born infants were eligible for participation. Infants with positive saliva screening results (rapid culture or PCR) were enrolled in the follow-up component of the study to monitor hearing outcome. Clinical decisions on evaluation and possible treatment of the CMV-infected infants were made by the physicians providing care for CMV-infected children at each study site. Institutional Review Board approval was obtained at each study site and written informed consent was obtained from parent(s). Race and ethnicity data were collected as self reported by parents. The study was designed by the CHIMES Study investigators in consultation with the project officers at the sponsoring institution, NIDCD. A previously described real-time PCR protocol was adapted to test newborn saliva specimens.¹⁴

Specimen Collection:

Saliva specimens were collected by swabbing the inside of the baby's mouth using a sterile polyester fiber-tipped applicator (PurFybr Inc., Munster, Ind.) and transported to the Central Laboratory at the University of Alabama at Birmingham within one week of collection.^{14, 19} During Phase I, saliva swabs were placed in TM and tested by rapid culture and liquid saliva PCR. During Phase II, an additional saliva swab collected at the same time was allowed to air dry, placed in a sterile tube without TM (“dried saliva swab”), and maintained and transported at ambient temperature to the Central Laboratory on a weekly basis. Since the collection of the dried saliva swab was phased in over several months, saliva specimens from some of the enrolled infants were tested using all three methods (rapid culture, liquid saliva PCR, and dried saliva PCR). The screening saliva specimens collected during Phase II were tested by rapid culture and dried saliva PCR only.

Specimen Processing:

Liquid saliva specimens were processed as described.^{14, 19} Dried saliva specimens were processed by adding 300 μl of PCR-grade water to the tubes containing saliva swabs, vortexing, and incubating for 20 minutes at room temperature. Five μL of the TM or the eluate containing saliva was used without a DNA extraction step for real-time PCR.

Detection of CMV in Saliva Specimens Using the Rapid Culture Assay:

A rapid culture assay for the detection of early antigen fluorescent foci using a monoclonal antibody against the major immediate early antigen of CMV was used to detect CMV in saliva specimens.^{14, 18, 19} Laboratory personnel performing the rapid culture were blinded to the results of PCR and vice versa.

Real-Time PCR of Saliva Specimens:

A real-time PCR protocol using the ABI 7500 Real Time PCR System (Applied Biosystems Inc., Foster City, Calif.) and utilizing Absolute Low ROX QPCR mix (ABGene USA, Rockford, Ill.) was performed to detect CMV DNA in saliva samples. The real-time PCR protocol was described in a recent report¹⁴, which is incorporated by reference herein to teach this method. Briefly, the following reagents were used: lyophilized Primers 80,000 μmol (ABI, Cat #4304971), first probe (described below) 50,000 μmol (ABI, Cat#4316032), second probe (described below) 50,000 μmol (ABI, Cat #450003), Absolute Low ROX QPCR mix (ABgene, Cat 4AB1318/13), molecular grade PCR water, 5-Prime (Fisher, Cat #955155017), optical PCR plates (ABgene, Cat #AB-1100/150), adhesive film for QPCR (ABgene, Cat #AB-1170), QPCR DNA standards at 10, 100, 1000, 10,000, and 100,000 copies/5 μL with target regions for both primer/probe sets. The final concentrations of primers and probe to be used in the PCR reaction were 900 nM and 200 nM, respectively. All samples were run in duplicate. Each panel contained a no-template control (NTC). Standard curves were derived from serial 10-fold dilutions of a positive control template (Calibrated Plasmid DNA). The positive control template was derived from cloning PCR target sequence into TOPO TA pCR2.1 (Invitrogen, CA) plasmid. The cycling parameters were as follows: Tag polymerase activation at 95° C. for 15 minutes, denaturation at 95° C. for 15 seconds, annealing/extension at 60° C. for 1 minute, all performed for 40 cycles; measurement of fluorescence was performed at the end of each annealing/extension cycle.

Two CMV primer sets from highly conserved target regions, one targeting the major envelope glycoprotein B (forward primer AGG TCTTCA AGO AAC TCA GCA AGA, reverse primer COG CAA TCG OTT TOT TOT AAA and probe 6FAM AAC CCC TCA. GCC ATT CTC TCG GC-TAMRA) and a second primer set from the IE-2 exon 5 gene (forward primer GAG CCC GAC ITF ACC ATC CA, reverse primer CAG CCG GCG GTA TCG A and probe VIC-ACC GCA ACA AGA TT-MGBNFQ) were included in the PCR, reaction. The reaction mixture contained primers at a concentration of 900 nM and the probe at 250 nM concentration. Each 25 μL reaction mixture contained 20 μL of master mix and 5 μL of test sample. Reactions were performed in duplicate and each plate contained plasmid standards incorporating both target sequences in 10-fold dilutions ranging between 100,000 and 10 genomic equivalents (ge) per reaction to generate standard curves. A sample was considered positive if ≧0.5 ge per reaction on the final PCR run.

Follow-up Testing:

Infants with positive rapid culture or PCR of screening saliva samples were reevaluated to determine whether the PCR-only positive screening results were true- or false-positive by testing saliva and urine specimens using rapid culture and PCR as described above.

Data Analysis:

The results of saliva PCR assays were compared with the rapid culture of saliva specimens obtained at birth. Saliva rapid culture at birth was considered the gold standard method in identifying infants with congenital CMV infection and sensitivity, specificity, and predictive values for the saliva real-time PCR assays were calculated using standard methods for proportions and exact 95% confidence limits. The likelihood ratios (LRs) are based on the ratio of sensitivity and specificity and are independent of the prevalence of congenital CMV infection in the population, therefore, LRs can be used directly to estimate the probability of congenital CMV infection at the individual level.²⁸ Positive LR was sensitivity/(1-specificity) and the negative LR was (1-sensitivity)/specificity and confidence intervals were determined using the method described by Simel and colleagues.²⁹ All statistical analyses were performed using SAS software version 9.2 (SAS Institute, Inc., Cary, N.C.).

Results

Study Population and Specimens:

During the study period, 34,989 infants were enrolled. The mean (SD) age at collection of saliva specimens for rapid culture and PCR was 1.0 (1.2) day. Characteristics of the study population are shown in Table 2. Infants were screened predominantly (98.0%) from the well baby nurseries. The median age for collection of follow-up samples was 3.6 weeks with an interquartile range of 2.6 to 6.6 weeks. Overall, 177 newborns (0.51%; 95% CI, 0.43-0.59%) tested positive for CMV on screening by rapid culture and/or PCR of saliva. No study-related adverse events were observed.

Newborn CMV Screening with Saliva Rapid Culture and Liquid Saliva Real-Time PCR:

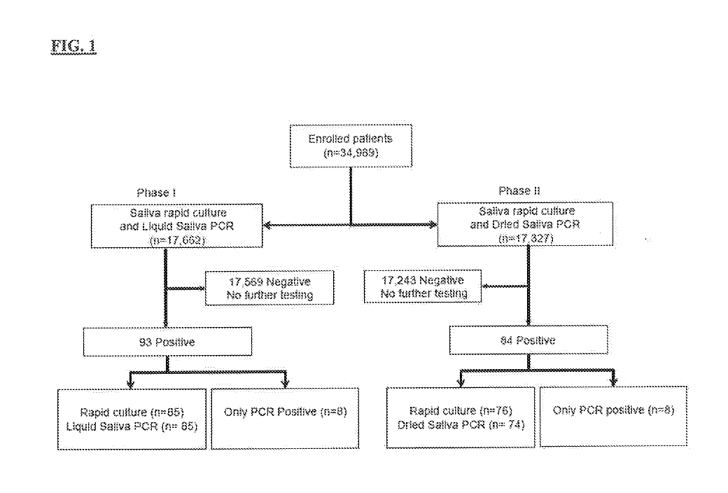

During Phase I, saliva specimens were collected from 17,662 newborns and tested for CMV using rapid culture and liquid saliva real-time PCR. Ninety-three (0.52%) infants tested positive for CMV by the saliva rapid culture or the PCR assay (FIG. 1). All infants positive by rapid culture (n=85) were also PCR-positive and PCR identified eight additional infants as infected (Table 3). The sensitivity and specificity of liquid saliva real-time PCR when compared to standard rapid culture were 100% (85/85, 95% CI, 95.8%-100%) and 99.9% (17569/17577, 95% CI, 99.9%-100%), respectively. The positive and negative predictive values for the saliva PCR assay were 91.4% (85/93, 95% CI, 83.8%-96.2%) and 100% (17569/17569, 95% CI, 99.9%100%), respectively. The positive LR for the liquid saliva PCR assay was 2197 (95% CI, 1099-4393) and the negative LR was 0 (95% CI, 0-0.1). Of the 93 newborns positive on screening, 79 (85%) infants were enrolled in follow-up. Among the 72 infants who were positive by both rapid culture and PCR and enrolled in follow-up, only one infant was negative on retesting. Of the eight PCR-only positive infants, seven were enrolled in follow-up; of those, six were negative by rapid culture and PCR of both saliva and urine specimens.

Newborn CMV Screening with Saliva Rapid Culture and Dried Saliva Real-Time PCR Assays:

During Phase II, a dried saliva specimen was also collected from 17,327 newborns. Of the 84 (0.49%) newborns who tested positive by either screening assay, the saliva rapid culture identified 76 (90%) infants as CMV-infected (FIG. 1). The dried saliva real-time PCR assay was positive for samples from 74 infants who tested positive by the rapid culture and an additional 8 newborns who were rapid culture negative (Table 3). When compared with saliva rapid culture, the sensitivity and the specificity of the dried saliva PCR assay were 97.4% (74/76, 95% CI, 90.8-99.7%) and 99.9% (17245/17253, 95% CI, 99.9-100%), respectively. The positive and negative predictive values for the dried saliva PCR were 90.2% (74182, 95% CI, 81.7-95.7%) and 99.9% (17243/17245, 95% CI, 99.9-100%), respectively. The positive LR for the dried saliva PCR assay was 2100 (95% CI, 1049-4202) and the negative LR was 0.03 (95% CI, 0.01-0.10) (Table 3). Of the 84 screen-positive infants, 74 (88%) were enrolled in follow-up. All 66 infants who were positive by both rapid culture and PCR and enrolled in follow-up were positive for CMV on retesting. The two infants who were positive by rapid culture but negative by PCR were positive for CMV on follow-up. Six of the 8 infants who were only PCR positive were retested; of those, four were negative and two were positive by rapid culture on follow-up.

Comparison between Liquid and Dried Saliva Real-Time PCR Assays:

All three screening methods (saliva rapid culture, liquid saliva PCR, and dried saliva PCR) were carried out on saliva specimens from 5,276 newborns. As shown in Table 4, there was excellent agreement between the liquid and the dried saliva PCR assays. Both liquid and dried saliva PCR assays identified 42 infants who were rapid culture positive and both identified one additional infant who was negative on screening saliva rapid culture and positive on follow-up testing.

Discussion

This large, prospective newborn CMV screening study demonstrates that the real-time PCR assay of both liquid and dried saliva samples has excellent sensitivity (>97%) and specificity (99.9%) when compared with the standard saliva rapid culture. Our findings show that the saliva PCR assays, which can easily be adapted for large-scale newborn screening, will identify most infants with congenital CMV infection.

The majority of infants with congenital CMV infection will not be identified by clinical examination during the newborn period. In addition, SNHL can develop after birth and continue to progress during early childhood in a significant proportion of children with CMV-associated SNHL.^{1, 6-8, 30} Thus, the availability of rapid and reliable diagnostic methods that can be adapted for high-throughput screening is essential for early identification of children at risk for CMV-associated SNHL. Testing DBS specimens using PCR-based methods appeared to be a promising strategy for newborn CMV screening because several previous studies reported that DBS PCR is highly sensitive in identifying infants with congenital CMV infection.^{15, 20, 21, 31} However, the results of our recent multicenter study comparing DBS real-time PCR assays with saliva rapid culture in more than 20,000 infants demonstrated that DBS PCR assays identified fewer than 40% of CMV-infected newborns.¹⁴ In addition, the performance of DBS PCR has been shown to vary with the size of the filter paper punch, the DNA extraction methods, and the PCR protocols employed by different investigators.^{16, 22, 23, 32} These findings, in addition to demonstrating the challenges in developing sensitive high-throughput assays for testing DBS, suggest that many newborns with congenital CMV infection may have detectable CMV DNA in peripheral blood. However, it is possible that further advances in PCR methodologies could improve the sensitivity of DBS PCR, allowing acceptable detection of infants with congenital CMV infection in the future.

Nevertheless, the data reported here show that the same DBS PCR protocol applied to saliva¹⁴ identified >97% of CMV-infected newborns. In addition, these findings demonstrate that saliva is a more reliable specimen than Dim for identifying congenital CMV infection by PCR and can be an effective tool for mass screening of newborns for CMV. Although testing of urine specimens collected using filter disks inserted into diapers of newborns was recently shown to be a promising approach for newborn CMV screening, urine specimen collection is not without challenges.^{17, 25} collecting urine specimens from all infants requires additional steps and time that are not needed for collecting saliva, making saliva advantageous for newborn screening. Further, validation of urine collection and urine PCR methods are needed before the practicality of this approach can be evaluated for large-scale newborn CMV screening.

In 16 infants, newborn saliva specimens were positive by real-time PCR but not rapid culture. To determine whether these represented false-positive results, infants who were positive on screening were retested using PCR of saliva and rapid culture of saliva and urine specimens obtained at the time of their enrollment into the follow-up study. If these tests were negative, we considered the screening results to be false positives. Three infants who were only positive by screening PCR at birth (one by liquid saliva PCR and two by dried saliva PCR) had positive rapid culture and PCR of follow-up specimens. These findings indicate that PCR assays identified additional CMV-infected newborns missed by rapid culture. False-positive PCR results were observed in ten infants who were PCR-positive (six by liquid saliva PCR assay and four by dried saliva PCR) but negative by rapid culture at birth, and whose follow-up saliva and urine specimens were negative for CMV. As CMV is occasionally shed in genital tract secretions of seropositive women at delivery and in breast milk of most seropositive mothers, these false-positive results could be due to virus-containing maternal secretions present in saliva samples.³³⁻³⁸ Although false-positive saliva PCR results could lead to unwarranted parental anxiety and additional testing of infants to confirm or exclude congenital CMV infection, the overall frequency of false-positive results for both liquid and dried saliva PCR assays was <0.03%. In addition, small negative likelihood ratios for both liquid and dried saliva PCR assays indicate that a negative result on these assays excludes congenital CMV infection (Table 3).²⁸ Nevertheless, when saliva PCR is employed as a newborn screening approach, a positive screening result should be confirmed within the first 3 weeks of age to avoid false-positive screening results.

The dried saliva PCR assay failed to detect two CMV-infected newborns leading to slightly lower sensitivity (97A %, 95% CI, 90.8%-99.7%) than the liquid saliva PCR. Nevertheless, the simplified specimen collection, storage, and transport procedures combined with high sensitivity of dried saliva PCR confirm this method as a reasonable newborn CMV screening approach. Although the need for collection of an additional specimen adds to the complexity of the existing newborn screening programs, the saliva PCR assays reported in this study have several advantages for newborn CMV screening. These include: (1) reasonable sensitivity and specificity, (2) non-invasive specimen collection, (3) elimination of the DNA extraction step, which simplifies the laboratory procedures providing significant cost savings, and (4) dried saliva specimens can be stored and transported at room temperature, further simplifying specimen handling and transport.

A limitation of this study is that the 34,812 infants negative by both rapid culture and PCR assays of screening saliva samples were not enrolled in follow-up to definitively exclude congenital CMV infection (by retesting using rapid culture of saliva or urine). Therefore, it is possible that CMV-infected newborns may have been missed by the rapid culture, affecting our determination of the sensitivity and specificity of saliva PCR. However, we believe this difference is negligible since the saliva rapid culture was demonstrated to be ≧98% sensitive.^{14, 19} At the present time, although imperfect, rapid culture of saliva or urine remains the most widely accepted standard method for identification of infants with congenital CMV infection.^{14, 19, 27}

In summary, the utility of saliva specimens for identification of CMV by PCR was demonstrated. The methods were further simplified by the use of dried specimens and processing with no DNA extraction step without significant loss of sensitivity and specificity. This strategy appears to be suitable for a high-throughput assay for large-scale screening to identify newborns with congenital CMV infection.

### REFERENCES

1. Dahle A J, Fowler K B, Wright J D, Boppana S B, Britt W J, Pass R E Longitudinal investigation of hearing disorders in children with congenital cytomegalovirus. J Am Acad Audioi 2000; 11:283-90.
2. Morton C C, Nance W E. Newborn hearing screening—a silent revolution. N. Engl. Med 2006; 354:2151-64.
3, Demmler G J. Infectious Diseases Society of America and Centers for Disease Control. Summary of a workshop on surveillance for congenital cytomegalovirus disease. Rev Infect Dis 1991; 13:315-29.
4. Stagno S. Cytomegalovirus. In: Remington J S, Klein J O, Wilson C B, Baker C J, eds. Injectious Diseases of the Fetus and Newborn Infant. Philadelphia, Pa.: W.B. Saunders Company; 2006:389-424.
5. Stehel E K, Shoup A G, Owen K E, et al. Newborn hearing screening and detection of congenital cytomegalovirus infection. Pediatrics 2008; 121:970-5.

6, Williamson W D, Demmler al, Percy A K, Catlin F L Progressive hearing loss in infants with asymptomatic congenital cytomegalovirus infection. Pediatrics 1992; 90:862-6.
7. Fowler K B, McCollister F P, Dahle A J, Boppana S, Britt W J, Pass R F. Progressive and fluctuating sensorineural hearing loss in children with asymptomatic congenital cytomegalovirus infection. J. Pediatr 1997; 130:624-30.
8. Foulon I, Naessens A, Foulon W, Casteels A, Gordts F. A 10-year prospective study of sensorineural hearing loss in children with congenital cytomegalovirus infection. J. Pediatr 2008; 153:84-8.
9. Hicks T, Fowler K, Richardson N I, Dahle A, Adams L, Pass R. Congenital cytomegalovirus infection and neonatal auditory screening. J Pediatr 1993; 123:779-82.
10. Joint Committee on Infant Hearing. Year 2007 Position Statement: Principles and Guidelines for Early Hearing Detection and Intervention Programs. Pediatrics 2007; 120:898-921.
11 Korver A M U, Konings S. Dekker F W, et al. Newborn hearing screening vs later hearing screening and developmental outcomes in children with permanent childhood hearing impairment. JAMA 2010; 304:1701-8.
12. Yamamoto A Y, Mussi-Pinhata M M, Marin I A Brito R M, Oliviera P F, Coelho T B. Is saliva as reliable as urine for detection of cytomegalovirus DNA for neonatal screening of congenital CMV infection? J Clin Viral 2006; 36:228-30.
13, Yamamoto A Y, Mussi-Pinhata M M, Pinto P C G, Figueriedo L T M, Jorge S M. Usefulness of blood and urine samples collected on filter paper in detecting cytomegalovirus by the polymerase chain reaction technique. J Virol Meth 2001; 97:159-0.64.
14. Boppana S B, Ross S A, Novak Z, et al. Dried blood spot real-time polymerase chain reaction assays to screen newborns for congenital cytomegalovirus infection. JAMA 2010; 303:1375-82.
15. Barbi M, Binda S, Primache V, et al. Cytomegalovirus DNA detection in Guthrie cards: a powerful tool for diagnosing congenital infection. J Clin Virol 2000; 17:159-65.
16. Soetens O, Vauloup-Fellous C, Foulon 1, et al. Evaluation of different cytomegalovirus (CMV) DNA PCR protocols for analysis of dried blood spots from consecutive cases of neonates with congenital CMV infections. J Clin Microbiol 2008; 46:943-6.
17. Nozawa N, Koyano S, Yamamoto Y, Inami Y, Kurarie 1, Inoue N. Real-time PCR assay using specimens on filter disks as a template for detection of cytomegalovirus in urine. J Clin Microbial 2007; 45:1305-7.
18. Boppana S B, Smith R, Stagno S, Britt W I Evaluation of a microtiter plate fluorescent antibody assay for rapid detection of human cytomegalovirus infections. J Clin Microbial 1992; 30:721-3.
19. Balcarek K B, Warren W, Smith R J, Lyon M D, Pass R F. Neonatal screening for congenital cytomegalovirus infection by detection of virus in saliva. J Infect Dis 1993; 167:1433-6.
20. Halwachs-Baumann G, Genser B, Pailer S, et al. Human cytomegalovirus load in various body fluids of congenitally infected newborns. J Clin Viral 2002; 25:S81-S7.
21. Revello M G, Gema G. Diagnosis and management of human cytomegalovirus infection in the mother, fetus, and newborn infant. Clin Micro Rev 2002; 15:680-715.
22. Bale J F, Jr. Screening newborns for congenital cytomegalovirus infection. JAMA 2010; 303:1.425-6.
23. Decks J J, Altman D G. Diagnostic tests 4: likelihood ratios, BMJ 2004; 329:168-9.
24. Simel D L, Samsa G P, Matchar D B. Likelihood ratios with confidence: sample size estimation for diagnostic studies. J Clin Epidemiol 1991; 44:763-70.
25. Dahle A J, McCollister F P, Stagno S, Reynolds D W, Hoffman H E. Progressive hearing impairment in children with congenital cytomegalovirus infection. J Speech Hear Disord 1979; 44:220-9.
26, Johansson Jonsson M, Ahlfors K, Ivarsson S A, Svanberg L, Guthenberg C. Retrospective diagnosis of congenital cytomegalovirus infection performed by polymerase chain reaction in blood stored on filter paper. Scand J Infect Dis 1997; 29:465-8.
27., Scanga L, Chiang 5, Powell C, et al. Diagnosis of human congenital cytomegalovirus infection by amplification of viral DNA from dried blood spots on perinatal cards. J Mol Diagn 2006; 8:240-5.
28. Yainagishi Y, Miyagawa B, Wada K, et al. CMV DNA detection in dried blood spots for diagnosing congenital CMV infection in Japan. J Med Viral 2006; 78:923-5.
29., Barbi M, MacKay W G, Binda S, van Loon A M. External quality assessment of cytomegalovirus DNA detection in dried blood spots. BMC Microbial 2008; 8:2,
30. de Vries J J C, Claas E C J, Kroes A C M, Vossen A C T M, Evaluation of DNA extraction methods for dried blood spots in the diagnosis of congenital cytomegalovirus infection. J Clin Viral 2009; 46:S37-42.
31, Gohring K, Dietz K, Hartleif S, Jahn C E, Hamprecht K. Influence of different extraction methods and PCR techniques on the sensitivity of HCMV-DNA detection in dried blood spot (DBS) filter cards. J Clin Viral 201.0; 48:278-81,
32. Inoue N, Koyano S. Evaluation of screening tests for congenital cytomegalovirus infection. Pediatr Infect Dis J 2008; 27:1824.
33, Dworsky M, Yow M, Stagno S, Pass R F, Alford C A. Cytomegalovirus infection of breast milk and transmission in infancy, Pediatrics 1983; 72:295-9.
34. Hamprecht K, Maschmann J, Vochem M. Dietz K, Speer C P, Jahn G. Epidemiology of transmission of cytomegalovirus from mother to preterm infants by breastfeeding. Lancet 2001; 357:513-18.
35. Pass R F, Stagno S, Dworsky M E, Smith R J, Alford C A. Excretion of cytomegalovirus in mothers: observation after delivery of congenitally infected and normal infants, J Infect Dis 1982; 146:1-6.
36, Reynolds D W, Stagno S, Hosty T S, Tiller M, Alford C A. Maternal cytomegalovirus excretion and perinatal infection. N Engl or Med 1973; 289:1-5,
37. Knox G E, Pass R E, Reynolds D W, Stagno 5, Alford C A. Comparative prevalence of subclinical cytomegalovirus and herpes simplex virus infections in the genital and urinary tracts of low income, urban females, J Infect Dis 1979; 140:419-22.
38. Mosca F, Pugni L, Barbi N I, Binda S. Transmission of cytomegalovirus. Lancet 2001; 357:1800.

TABLE 2
Characteristics of 34,989 newborns that were tested
by rapid culture and real-time PCR assays of saliva
specimens for congenital CMV infection.

| Characteristic | No. (%) |
|---|---|
| Gender | |
| Female | 17,278 (49.4) |
| Male | 17,711 (50.6) |
| Race & Ethnicity | |
| Asian | 1358 (3.9) |
| Black | 8,298 (23.7) |
| White, Hispanic | 11,356 (32.5) |
| White, Non-Hispanic | 12,835 (36.7) |
| Other, including more than one race | 1,142 (3.3) |
| Insurance for Hospital Stay | |
| Private | 23,326 (66.7) |
| Public or No Insurance | 11,663 (33.3) |
| Hospital Nursery | |
| Well Baby | 34,275 (98.0) |
| Neonatal Intensive Care | 714 (2.0) |
| Maternal Age | |
| Mean (SD) | 27.3 (6.1) |
| Median (range) | 27 (12-52) |

TABLE 3
Utility of real-time PCR assays of liquid and dried saliva specimens in identifying
infants with congenital CMV infection.

| | Liquid Saliva PCR | | | Dried Saliva PCR | | |
|---|---|---|---|---|---|---|
| | (June, 2008- | | | (March, 2009- | | |
| | February, 2009) | | | November, 2009) | | |
| Saliva Rapid Culture | Positive | Negative | Total | Positive | Negative | Total |
| Positive | 85 | 0 | 85 | 74 | 2 | 76 |
| Negative | 8 | 17,569 | 17,577 | 8 | 17,243 | 17,251 |
| Total | 93 | 17,569 | 17,662 | 82 | 17,245 | 17,327 |

| | | | | |
|---|---|---|---|---|
| Sensitivity | 100% | (95.8-100%) | 97.4% | (90.8-99.7%) |
| Specificity | 99.9% | (99.9-100%) | 99.9% | (99.9-100%) |
| Positive Likelihood Ratio | 2197 | (1099-4393) | 2100 | (1049-4202) |
| Negative Likelihood Ratio | 0 | (0-0.1) | 0.03 | (0.01-0.10) |
| Positive Predictive Value | 91.4% | (83.8-96.2%) | 90.2% | (81.7-95.7%) |
| Negative Predictive Value | 100% | (99.9-100%) | 99.9% | (99.9-100%) |

PCR, polymerase chain reaction;
CMV, cytomegalovirus

TABLE 4
Comparison of saliva rapid culture, liquid saliva PCR and dried
saliva PCR assays in 5,270 infants who were tested by all three
screening assays for the detection of congenital CMV infection.

| | Liquid Saliva PCR | | Dried Saliva PCR | | |
|---|---|---|---|---|---|
| Saliva Rapid Culture | Positive | Negative | Positive | Negative | Total |
| Positive | 42 | 0 | 42 | 0 | 42 |
| Negative | 1 | 5233 | 1 | 5233 | 5234 |
| Total | 43 | 5233 | 43 | 5233 | 5276 |

PCR, polymerase chain reaction; CMV, cytomegalovirus

1. A method of detecting a virus in a saliva sample, the method comprising:
(a) contacting the sample with a first forward amplification primer and a first reverse amplification prim r said primers capable of hybridizing to sequences flanking a target sequence;
(b) contacting the sample with a second forward amplification primer and a second reverse amplification primer, said second primers capable of hybridizing to sequences flanking a second target sequence, to form a reaction mixture;
(b) performing the polymerase chain reaction on the reaction mixture to form an amplicon; and
(c) detecting the target sequence in the amplicon.

2. The method of claim 1, wherein the virus is a cytomegalovirus (CMV), wherein at least one target sequence is selected from the group consisting of a consensus sequence among all CMV, a consensus sequence of at least one strain of CMV, and a sequence unique to CMV.

3. The method of claim 2, wherein the sample is not subject to DNA extraction prior to performing the polymerase chain reaction.

4. The method of claim 1, wherein the saliva sample is a liquid sample.

5. The method of claim 1, wherein the saliva sample is a dried sample.

6. The method of claim 1, wherein the sample is a saliva sample from a human neonate.

7. The method of claim 1, wherein at least one of the first or second targets is the major envelope glycoprotein B gene or a portion thereof.

8. The method of claim 7, wherein the forward primer comprises SEQ ID NO: 1 and the reverse primer comprises SEQ ID NO: 2.

9. The method of claim 7, wherein detecting the target sequence comprises contacting the amplicon to a probe, and wherein the probe comprises the nucleic acid sequence SEQ ID NO: 3.

10. The method of claim 1, wherein at least one of the first or second targets is the immediate early region 2 exon 5 gene or a portion thereof.

11. The method of claim 10, wherein the forward primer comprises SEQ ID NO: 4 and the reverse primer comprises SEQ ID NO: 5.

12. The method of claim 10 wherein detecting the target sequence comprises contacting the amplicon to a probe, and wherein the probe comprises the nucleic acid sequence SEQ ID NO: 6.

13. The method of claim 1, wherein detecting the target sequence comprises contacting the amplicon to a probe.

14. The method of claim 1, wherein the polymerase chain reaction is a quantitative real-time polymerase chain reaction.

15. A method of detecting a virus in a saliva sample, the method comprising:
(a) contacting the sample with a first forward amplification primer and a first reverse amplification primer, said primers capable of hybridizing to sequences flanking a first target sequence, to form a reaction mixture;
(b) performing the polymerase chain reaction on the reaction mixture to form an amplicon; and
(c) detecting the target sequence in the amplicon;
wherein the sample is not subject to DNA extraction prior to performing the polymerase chain reaction.

16. The method of claim 15, wherein the virus is a cytomegalovirus (CMV), wherein the target sequence is selected from the group consisting of a consensus sequence among all CNN, a consensus sequence of at least one strain of CMV, and a sequence unique to CMV.

17. The method of claim 15, wherein the saliva sample is a liquid sample.

18. The method of claim 15, wherein the saliva sample is a dried sample.

19. The method of claim 15, Wherein the sample is a saliva sample from a human neonate.

20. The method of claim 15, wherein at least one of the first or second targets is the major envelope glycoprotein B gene or a portion thereof.

21. The method of claim 20, wherein the forward primer comprises SEQ ID NO: 1 and the reverse primer comprises SEQ ID NO: 2.

22. The method of claim 20, wherein detecting the target sequence comprises contacting the amplicon to a probe, and wherein the probe comprises the nucleic acid sequence SEQ. ID NO: 3.

23. The method of claim 15, wherein at least one of the first or second targets is the immediate early region 2 exon 5 or a portion thereof.

24. The method of claim 23, wherein the forward primer comprises SEQ ID NO: 4 and the reverse primer comprises SEQ ID NO: 5.

25. The method of claim 2.3 wherein detecting the target sequence comprises contacting the amplicon to a probe, and wherein the probe comprises the nucleic acid sequence SEQ ID NO: 6.

26. The method of claim 15, wherein detecting the target sequence comprises contacting the amplicon to a probe.

27. The method of claim 15, wherein the polymerase chain reaction is quantitative real-time polymerase chain reaction.

28. A method of detecting a virus in a saliva sample, the method comprising:
(a) contacting the sample with a first forward amplification primer and a first reverse amplification primer, said primers capable of hybridizing to sequences flanking a consensus sequence comprising SEQ ID NO: 8 or a portion of SEQ ID NO: 8;
(b) contacting the sample with a second forward amplification primer and a second reverse amplification primer, said second primers capable of hybridizing to sequences flanking a consensus sequence comprising SEQ ID NO: 7 or a portion SEQ ID NO: 7, to form a reaction mixture;
(c) performing real-time polymerase chain reaction on the reaction mixture to form an amplicon;
(d) contacting the amplicon with a probe comprising SEQ ID NO: 4;
(e) contacting the amplicon with a probe comprising SEQ ID NO: 6 and
(f) detecting any hybridized probe.

29. (canceled)

30. A kit for the detection of a virus in saliva by real-time PCR, the kit comprising a first forward primer, a first reverse primer, a DNA polymerase, and not including reagents for DNA extraction.

31-48. (canceled)

49. A method of diagnosing a subject for CMV infection, comprising performing the method of claim 1 on a saliva sample from the subject, and concluding that the subject suffers from CMV infection if CMV is detected in the sample.


SEQUENCE LISTING (10 sequences)

SEQ ID NO: 1 - DNA, 24 bp, Artificial Sequence
  PCR Primer
aggtcttcaaggaactcagcaaga

SEQ ID NO: 2 - DNA, 21 bp, artificial sequence
  PCR Primer for Glc-B AD-1
cggcaatcggtttgttgtaaa

SEQ ID NO: 3 - DNA, 23 bp, artificial sequence
  Synthetic probe
aacccgtcagccattctctcggc

SEQ ID NO: 4 - DNA, 20 bp, artificial sequence
  Synthetic PCR primer
gagcccgactttaccatcca

SEQ ID NO: 5 - DNA, 16 bp, artificial sequence
  Synethetic PCR primer
cagccggcggtatcga

SEQ ID NO: 6 - DNA, 14 bp, artificial sequence
  Synthetic prrobe
accgcaacaagatt

SEQ ID NO: 7 - DNA, 500 bp, Human cytomegalovirus
  misc_feature (43)..(45): In some strains there may be a gap at one or more of these positions as opposed to the noted base
ctcggactcactctccgagtccgaagccgaactgcaggaggaggaagacgaagaggaactatcttcatcgggycggsccarrggatcgggaagaggagggtggttcatctgggagagcggrtgcgtgggagaggtcactcgcggcgtgccgctgccggtggaaggggaagacgcggtrgcaccgcgggtytcgacttcttcaccctgttcttcctcgctatcagagatcacgatacagccggcggtatcgataatcttgttgcggtactggatggtaaagtcgggctcgggcttgatgtcttcctgtttgatsaggggcagcatgataggcgcgggaggcacgggcggtttaataatcaccttgaaaggacgcgtggttttgcgcggtttcttacgcgggctgagctcgggagtagcggatgccccggggakaggagtgttagtaaccgcgacgctggtgggggtcggcttgttaagaggggcgctgctaacgctgcaag

SEQ ID NO: 8 - DNA, 500 bp, Human cytomegalovirus
  misc_feature (1)..(206): AD-1 region
tccacrtaytcgtaggccgagttsccggcgatgaagatcttgaggctgggaarctgacattcctcagtgcggtggttgcccaacargatttcgttrtcctcrcccagttgaccgtactgcacgtacgagctgttgrcraaattaaagatgaccackggtcgtgagtagcagcgtcctggcgaytcyttcacrttcatatcacgcagcaccttracgctkgtttggttratrgtcacrcagctggccarrcccarracatcacccatgaaacgcgcggcaatcggtttgttgtaratggccgagagaatrgctgayggrttgatcttgctragttycytgaagacctctagggtgcgccgttgatccacacaccargcytckgcgatytgcgccarcgcccgrttgatgtarcygcgcaacgtrtcataggtgaactgcagctgrgcgtagascasattkygyacmgaytcsmtkyyrrryramwgrgtkgymktrttgyc

SEQ ID NO: 9 - DNA, 500 bp, human cytomegalovirus
ctcggactcactctccgagtccgaagccgaactgcaggaggaggaagacgaagaggaactatcttcatcgggccggcccaagggatcgggaagaggagggtggttcatctgggagagcgggtgcgtgggagaggtcactcgcggcgtgccgctgccggtggaaggggaagacgcggtagcaccgcgggtttcgacttcttcaccctgttcttcctcgctatcagagatcacgatacagccggcggtatcgataatcttgttgcggtactggatggtaaagtcgggctcgggcttgatgtcttcctgtttgatgaggggcagcatgataggcgcgggaggcacgggcggtttaataatcaccttgaaaggacgcgtggttttgcgcggtttcttacgcgggctgagctcgggagtagcggatgccccggggagaggagtgttagtaaccgcgacgctggtgggggtcggcttgttaagaggggcgctgctaacgctgcaag

SEQ ID NO: 10 - DNA, 500 bp, Human Cytomegalovirus
tccacgtactcgtaggccgagttcccggcgatgaagatcttgaggctgggaagctgacattcctcagtgcggtggttgcccaacaggatttcgttgtcctcgcccagttgaccgtactgcacgtacgagctgttggcgaaattaaagatgaccacgggtcgtgagtagcagcgtcctggcgaytccttcacgttcatatcacgcagcaccttgacgctggtttggttgatggtcacgcagctggccaggcccaagacatcacccatgaaacgcgcggcaatcggtttgttgtaaatggccgagagaatggctgacgggttgatcttgctgagttccttgaagacctctagggtgcgccgttgatccacacaccaggcttctgcgatttgcgccagcgcccggttgatgtaaccgcgcaacgtgtcataggtgaactgcagctgggcgtagaccagattgtgcaccgattccatgttggacaaatgagttgtattrttgyc